(19) United States
(12) Patent Application Publication
(10) Pub. No.: US 2021/0388594 A1
(43) Pub. Date: Dec. 16, 2021

(54) Toilet with a Multi Function Bidet Wand

(71) Applicant: Medic, Inc., Provo, UT (US)

(72) Inventors: David R. Hall, Provo, UT (US); Joshua Larsen, Spanish Fork, UT (US); Jared Reynolds, Spanish Fork, UT (US)

(73) Assignee: Medic, Inc., Provo, UT (US)

(21) Appl. No.: 16/902,070

(22) Filed: Jun. 15, 2020

(51) Int. Cl. E03D 9/08; E03D 9/10; G01N 33/483; G01N 1/24; G01J 5/10; A47K 5/12; H04N 5/225; G06T 7/00; A61B 5/00; A61M 35/00; A61B 10/00; A61B 5/20; A61B 8/08

(52) U.S. Cl. CPC E03D 9/085; G01J 2005/0077; G01N 33/4833; G01N 1/24; G01J 5/10; A47K 5/12; H04N 5/2253; G06T 7/0012; A61B 5/0077; A61M 35/20; A61B 10/0038; A61B 5/445; A61B 5/208; A61B 5/489; A61B 8/085; E03D 9/10

(57) ABSTRACT

A toilet comprising a seat for supporting a user during use of the toilet, a bowl for catching excreta from the user, a retractable bidet wand adapted to extend after excretion has concluded, a bidet head located on the end of the bidet wand, adapted to spray water on the exposed surface of the user, an image sensor on the bidet wand, adapted to capture images of the exposed surfaces of the user, and a processor to analyze the images to detect health issues.

### CROSS-REFERENCE TO RELATED APPLICATIONS

This application claims the benefit under 35 U.S.C. 119(e) of U.S. Provisional Patent Application Nos. 62/862,605 filed Jun. 17, 2019. The disclosure of the application is hereby incorporated herein by reference.

### TECHNICAL FIELD

The present disclosure relates to toilets. More particularly, it relates to toilets equipped with a multi-function bidet wand.

### BACKGROUND

The ability to track an individual's health and wellness is currently limited due to the lack of available data related to personal health. Many diagnostic tools are based on examination and testing of excreta, but the high cost of frequent doctor's visits and/or scans make these options available only on a very limited and infrequent basis. Thus, they are not widely available to people interested in tracking their own personal wellbeing.

Toilets present a fertile environment for locating a variety of useful sensors to detect, analyze, and track trends for multiple health conditions. Locating sensors in such a location allows for passive observation and tracking on a regular basis of daily visits without the necessity of visiting a medical clinic for collection of samples and data. Monitoring trends over time of health conditions supports continual wellness monitoring and maintenance rather than waiting for symptoms to appear and become severe enough to motivate a person to seek care. At that point, preventative care may be eliminated as an option leaving only more intrusive and potentially less effective curative treatments. An ounce of prevention is worth a pound of cure.

Some toilets include a bidet wand to deliver a stream of water to wash the genitalia area of a user after an excretion event. The bidet wand comes in close proximity to exposed surfaces of a user. The wand may thus comprise further capabilities to improve the health and wellness of a user without the need for a visit to a health care specialist. This may further motivate a user to use a bidet with capabilities to detect genitalia-related health issues who may otherwise be too embarrassed to expose themselves to a nurse, doctor, or other health care specialist.

### SUMMARY

One aspect of the present invention, a toilet comprises a seat for supporting a user during use of the toilet, a bowl for catching excreta from the user, a retractable bidet wand adapted to extend after excretion has concluded, a bidet head located on the end of the bidet wand, adapted to spray water on the exposed surface of the user, an image sensor on the bidet wand, adapted to capture images of the exposed surfaces of the user, and a processor to analyze the images to detect health issues.

In another aspect, the bidet head is adapted to aim pressurized jets of water to remove debris from the bowl. The bidet head can also be adapted to aim pressurized jets of water at the excreta to comminute the excreta

In still another aspect, the bidet head can be adapted to capture gases in the bowl for analysis following excretion by a user.

In a still further aspect, the bidet head can be adapted to capture images of the excreta.

In a yet still further aspect, the image sensor can be a charge coupled device (CCD) or a complementary metal-oxide semiconductor (CMOS) sensor.

In still yet another aspect of the invention, the bidet wand and bidet head are capable of rotating around a longitudinal axis.

In another aspect, the bidet head comprises an infrared (IR) camera. The infrared camera is adapted to identify heat differentials in the exposed surfaces of the user.

In still another aspect, the bidet head can be adapted to emit a health and wellness spray towards the exposed surface of a user. The health and wellness spray can include one or more ointments, lubricants, herbals, medicinal drugs, pain killers, topicals, essential oils, soaps, foams, cleansers, surfactants, moisturizers, deodorants, dyes, conditioners, or a combination thereof

In a still further aspect, the bidet head can be adapted to emit a stream of air to dry the exposed surface of a user.

In a yet still further aspect, the bidet head can be adjustable to direct water or a health and wellness spray to the genitalia area of a male or female user.

In still yet another aspect of the invention, the bidet head can be adapted to detect a fecal biomarker that is enhanced by ingestion of a dye, tracer, or marker by a user before excreting a sample of feces.

In another aspect of the present invention, the bidet head can be adapted to spray a dye, tracer, or marker onto the surface of a sample of excreta to detect one or more biomarkers.

In another aspect, the bidet head further includes one or more image sensors capable of forming a three-dimensional image of the exposed surfaces of the user.

In still another aspect, the bidet head further includes a light emitter to illuminate the exposed surface of a user.

In still yet another aspect, the bidet head includes an ultra-violet (UV) light emitter to disinfect the bowl.

In a still further aspect, the bidet head further includes a self-cleaning device.

In still yet another aspect of the invention, the bidet head can be adapted to heat an emitted liquid.

Further aspects and embodiments are provided in the foregoing drawings, detailed description and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1A illustrates an analytical toilet with the lid closed, according to an embodiment of the disclosure.

FIG. 1B illustrates an analytical toilet with lid open, according to an embodiment of the disclosure.

FIG. 1C illustrates an overhead view of an analytical toilet with lid open, according to an embodiment of the disclosure.

FIG. 2A illustrates a cross-sectional view of a portion of an analytical toilet equipped with a bidet wand and bidet head, according to an embodiment of the disclosure.

FIG. 2B illustrates a cross-sectional view of a portion of an analytical toilet in operation, according to an embodiment of the disclosure.

FIG. 2C illustrates a cross-sectional view of a portion of an analytical toilet in operation, according to an embodiment of the disclosure.

FIG. 2D illustrates a cross-sectional view of a portion of an analytical toilet in operation, according to an embodiment of the disclosure.

### DETAILED DESCRIPTION

### Overview

Embodiments of methods, materials and processes described herein are directed towards analytical toilets. Analytical toilets are equipped to provide health and wellness information related to excreta deposited by a user.

Analytical toilets can be equipped with a bidet wand. The disclosure herein describes a system wherein the bidet wand further comprises a bidet head and is capable of multiple functions to improve the health and wellness of a user.

### Definitions

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “toilet” is meant to refer to any device or system for receiving human excreta, including urinals. This includes a conventional toilet upon which a user sits (“western style”), as well as toilet over which a user squats (“eastern style”) and before which a user stands (“urinal”).

As used herein, the term “bowl” refers to the portion of a toilet that is designed to receive excreta.

As used herein, the term “user” refers to any individual who interacts with the toilet and deposits excreta therein.

As used herein, the term “excreta” refers to any substance released from the body including urine, feces, menstrual discharge, and anything contained or excreted therewith.

As used herein, the term “bidet” is meant to have a relatively broad meaning, referring to any wand or other structure that is used with a toilet to wash the genital and anal area of a user of the toilet. The modern bidet includes a plumbed-in water supply and a drainage opening and is thus a type of plumbing fixture subject to local hygiene regulations. The bidet is designed to promote personal hygiene and is used after an excretion event by a user.

As used herein, the term “sensor” is meant to refer to any device for detecting and/or measuring a property of a person or of a substance regardless of how that property is detected or measured, including the absence of a target molecule or characteristic. Sensors may use a variety of technologies including, but not limited to, MOS (metal oxide semiconductor), CMOS (complementary metal oxide semiconductor), CCD (charge-coupled device), FET (field-effect transistors), nano-FET, MOSFET (metal oxide semiconductor field-effect transistors), spectrometers, spectrophotometer, colorimeter, refractometer, volume measurement devices, weight sensors, temperature gauges, chromatographs, mass spectrometers, IR (infrared) detector, near IR detector, visible light detectors, and electrodes, microphones, load cells, pressure gauges, PPG (photoplethysmogram), thermometers (including IR and thermocouples), rheometers, durometers, pH detectors, scent detectors gas, and analyzers.

As used herein, the term “imaging sensor” is meant to refer to any device for detecting and/or measuring a property of a person or of a substance that relies on electromagnetic radiation of any wavelength (e.g., visible light, infrared light, ultra-violet, x-ray) or sound waves (e.g., ultrasound) to view the surface or interior of a user or substance. The term “imaging sensor” does not require that an image or picture be created or stored even if the sensor is capable of creating an image.

### Exemplary Embodiments

The present disclosure describes a bidet comprising an analytical toilet and a multi-functional bidet wand and bidet head. The bidet wand can extend, retract, and rotate. The bidet head includes an illumination source and a camera to facilitate the capture of three-dimensional (3D) images of the exposed surface of a user.

The illumination source can emit variable intensity and frequency light to identify and collect health and wellness information on the exposed surface of the user. The health and wellness information includes information on skin sores, skin rashes, skin swelling, vascular health, and other trends. Infrared (IR) cameras and sensors may be used to identify skin temperature differentials (e.g., “hot spots”).

The bidet head can emit one or more fluidic sprays/mists specifically on detected skin sores, rashes, swelling or other skin issues identified by an IR camera or sensor to provide treatment and comfort to the user. The bidet head may also emit one or more fluidic ointments, lotions, lubricants, conditioners, essential oils, deodorants, topical solutions, soaps, moisturizers, or other materials over a larger area of exposed surfaces of a user. The bidet head may also emit a stream of air to aid in drying the emitted fluidic sprays or mists.

The bidet head can be used to emit a dye, tracer or marker onto excreta left by a user for analysis.

Analytical Toilet with Multi-Functional Bidet Wand

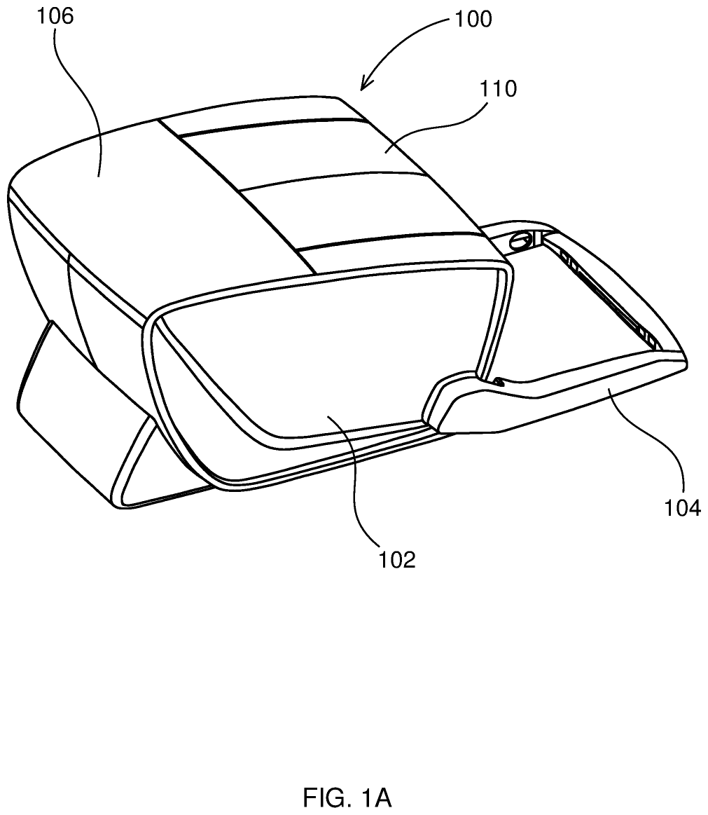

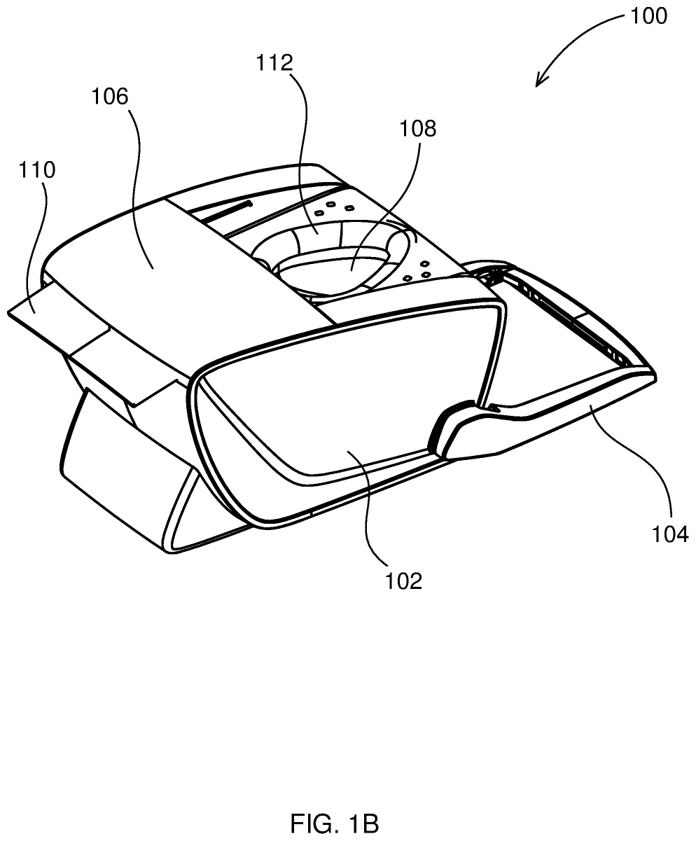

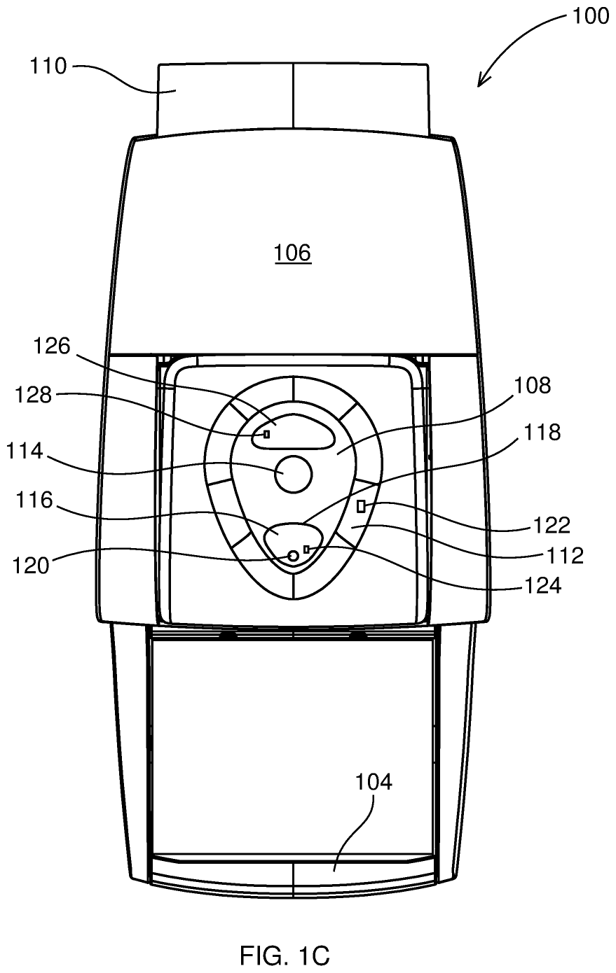

Now referring to FIGS. 1A-C, a preferred embodiment of an analytical toilet 100 is shown. FIG. 1A illustrates the analytical toilet 100 with the lid 110 closed, according to an embodiment of the disclosure. FIG. 1A further shows exterior shell 102, foot platform 104 and rear cover 106. The lid 110 is closed to prevent a user from depositing excreta in toilet 100 until the toilet is ready for use.

FIG. 1B illustrates toilet 100 with lid 110 open, according to an embodiment of the disclosure. Toilet 100 includes exterior shell 102, rear cover 106, bowl 108, seat 112, lid 110, and foot platform 104. Housed within toilet 100 are a variety of features, including equipment, that facilitate receiving excreta, processing urine for analysis, analyzing urine, and disposing of urine and any feces. FIG. 1B shows toilet 100 with lid 110 open so a user can sit on seat 112 and deposit excreta in toilet 100. Bowl 108 may be transparent, translucent, or opaque.

FIG. 1C illustrates an overhead view of an analytical toilet 100 with lid 110 open, according to an embodiment of the disclosure. Toilet 100 further includes a drain 114 where feces or urine may be removed and directed to a sewer or other location. Toilet 100 further includes a urine collection area 116 that is separated from the rest of bowl 108 by a partition 118. Urine collection 116 area further comprises a urine collection port 120. Port 120 may be a hole or a slit or other shape. Port 120 may comprise a filter or screen to prevent solid material, such as feces or toilet paper, from entering the port 120. The seat 112 may comprise one or more sensors 122. In some embodiments, there may be no sensors in the seat 112. The one or more sensors 122 may be located anywhere on the seat 112. Sensors 122 may be configured to measure body weight, body temperature, skin moisture, pulse, or other information about a user. The urine collection area 116 further comprise one or more sensors 124. Sensors 124 in urine collection area 116 may be used to determine temperature, detect solids, or other information about the urine from a user. In some instances, urine collection area 116 may not have one or more sensors.

Toilet 100 comprises a fecal shelf 126. Fecal shelf 126 is an area where feces can be received from a user, collected and analyzed. Fecal shelf 126 comprises one or more sensors, such as imaging sensors 128. The one or more imaging sensors 128 may be used to determine the shape or the form of the feces, detect parasites, blood, or other information.

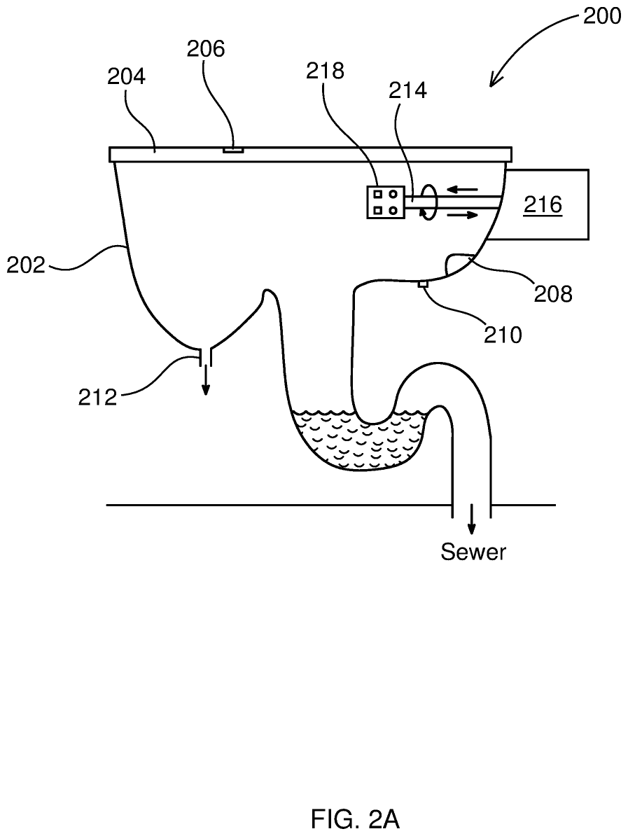

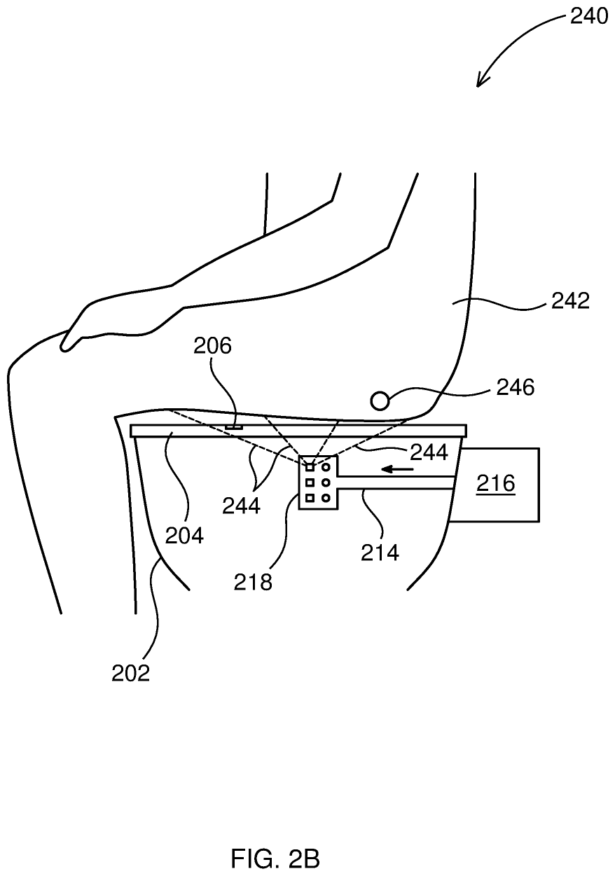

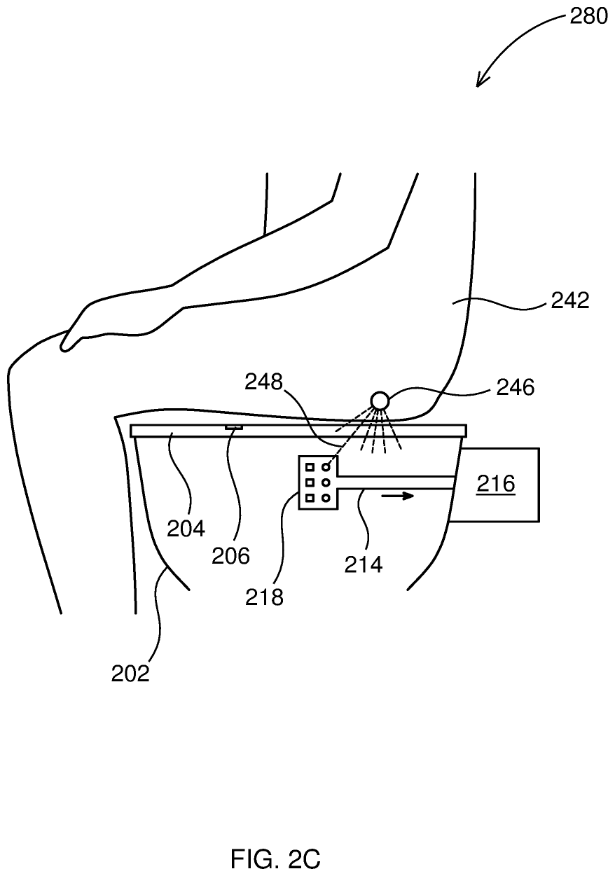

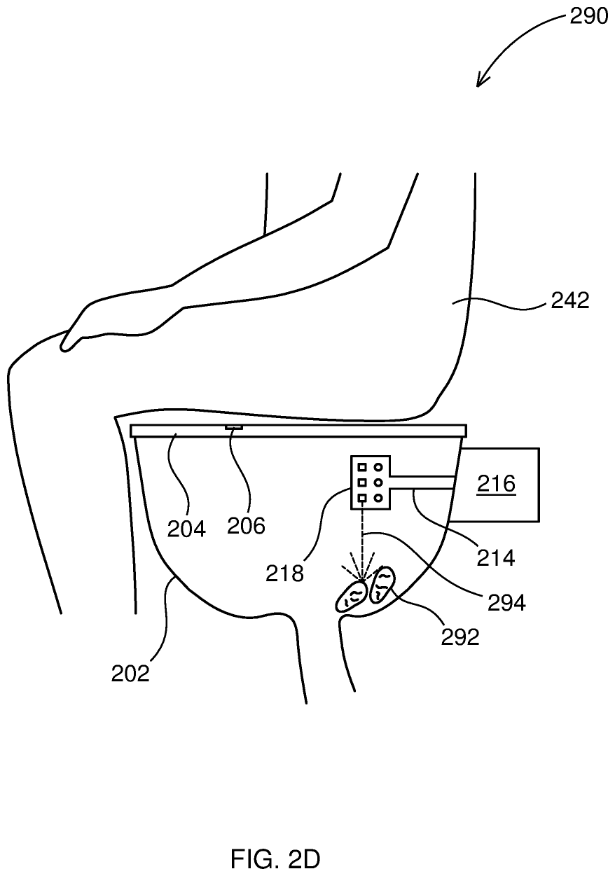

FIG. 2A illustrates a cross-sectional view of a portion of an analytical toilet 200 equipped with a bidet wand and bidet head, according to an embodiment of the disclosure. The cover, exterior shell and other components have been omitted for clarity. Toilet 200 comprises a bowl 202, seat 204, seat sensor 206, and fecal shelf 208. Toilet 200 further comprises an optional fecal sensor 210 and urine port 212. Urine may drain through port 212 towards an optional detection system for further analysis.

Toilet 200 further comprises a bidet wand 214. Bidet wand 214 can be extendable, retractable and may rotate in a circular manner around a longitudinal axis. Bidet wand 214 further comprises a drive motor system to power and control the movement of the wand. Bidet wand 214 further comprises a bidet head 218. The bidet head 218 may be located on the end of the bidet wand 214 or any other location. The bidet wand 214 and head 218 may be adjustable based on the sex of the user for more targeted cleaning and washing of the genitalia area. Head 218 may comprise one or more gas inlets. Gas inlets are used to collect gases from the bowl using a gas suction device. The gases may then be directed to a gas sensor device, such as a gas chromatograph (GC) to determine the chemical composition of the gases.

Head 218 on bidet wand 214 comprises one or more liquid dispensers. Liquids may be dispensed in a stream, spray, or mist form onto the exposed surface of a user. The liquid may be water to clean the genitalia area of a user after an excretion event. The liquids may be a health and wellness substance such as one or more of an ointment, lubricant, herbal, medicinal drug, pain killer, water, aqueous solution, topical solution, essential oil, soap, foam, cleanser, surfactant, moisturizer, deodorant, dye, or a conditioner. The medicinal drug may include an anti-microbial, such as a triple anti-biotic comprised of neomycin sulfate, bacitracin zinc, and polymyxin-B sulfate. The composition may be 5 mg of neomycin sulfate, 400 units of bacitracin zinc, and 5000 units of polymyxin-B sulfate. The medicinal drug may be lidocaine, such as a 5% by weight ointment of lidocaine. In some instances, the head 218 may heat the emitted fluid for a more comfortable experience by a user.

In some embodiments, the bidet head 218 is adapted to aim pressurized jets of water at the excreta to comminute or macerate the excreta. The pressurized jets of water may be used to remove loose or adhered debris from the bowl.

Head 218 on bidet wand 214 may emit a stream of air. The air may be used to dry the user after a liquid has been dispensed onto an exposed surface of a user. The air may be compresses air with a pressure in the range of about 1-50 psi.

Head 218 on bidet wand 214 may comprise a light emitter. The light emitter emits electromagnetic radiation in the visible light range (^{˜}390-700 nm) and may be used to illuminate the exposed surface of a user. The light emission source may be a light emitting diode (LED), fluorescent lamp, high-intensity discharge lamp, halogen lamp, or an incandescent light source. Illumination of the exposed surface may improve imaging of the surface.

Head 218 on bidet wand 214 comprises one or more cameras. The one or more cameras may be used to take two or three-dimensional images of the exposed surface of a user while sitting on the seat of an analytical toilet. The one or more cameras may take still or video images. The images may be digital images.

Head 218 on bidet wand 214 may comprise a thermographic camera, such as an infrared (IR) camera. The IR camera utilizes IR radiation in the wavelength range of about 700 nm to about 1 mm. The IR camera can be used to detect “hot spots” on the exposed surface of a user. The hot spots include rashes, abrasions, sores, blisters, infections, or other skin disorders. Once a target is detected by the IR camera, the bidet head 218 may be used to dispense a liquid onto the hot spot to treat and comfort the user. The bidet head 218 may dispense a fluidic material on a localized spot as directed by the IR camera, such as a sore, or over a larger surface area such as to treat a rash. An IR camera can also be used to monitor general blood flow through the genitalia area of a user and other health and wellness indicators.

Head 218 on bidet wand 214 may comprise one or more imaging sensors to image the exposed surface of a user. The imaging sensors may be a charge coupled device (CCD) or a complementary metal-oxide semiconductor (CMOS) sensor. Head 218 and bidet wand 214 may comprise other sensors and capabilities.

In various exemplary embodiments, the one or more imaging sensors located in the bidet head 218 may include one or more near infrared (NIR) sensitive cameras and a source of NIR light. By illuminating veins on an exposed surface of a user with NIR light, the veins located just below the skin can be detected, evaluated, and tracked. Data gathered over time about veins allows one to track trends and detect changes in size and other characteristics, including varicose veins. This information may be used to monitor indicators for potential blood clots, blood flow, and general vein health.

In various exemplary embodiments, the one or more imaging sensors include one or more ultrasonic 3D imaging devices. Ultrasound technology may be used to create images of the genitalia area of a male or female user. For example, the imaging sensors may be used to detect protrusions such as hemorrhoids.

In some embodiments, the one or more imaging sensors may detect a dirty genitalia area of a male or female user even after a portion of water has been emitted from the bidet head 218. A second portion of water may then be emitted from the bidet head 218 to further clean the genitalia area. This process may be repeated until the genitalia are of the user is sufficiently cleaned.

The one or more imaging sensors in bidet head 218 may be adapted to monitor the condition of the exposed surface and genitalia area of a user by looking at blood flow, vein condition, swelling (such as hemorrhoids), protrusions, potential blood clots, and skin texture among others.

Various control mechanisms for the invention described herein may be implemented fully or partially in software and/or firmware. This software and/or firmware may take the form of instructions contained in or on a non-transitory computer-readable storage medium. Those instructions may then be read and executed by one or more processors to enable performance of the operations described herein, such as processing the data from one or more imaging sensors or cameras located in bidet head 218. The instructions may be in any suitable form, such as but not limited to source code, compiled code, interpreted code, executable code, static code, dynamic code, and the like. Such a computer-readable medium may include any tangible non-transitory medium for storing information in a form readable by one or more computers, such as but not limited to read only memory (ROM); random access memory (RAM); magnetic disk storage media; optical storage media; a flash memory, etc.

Data collected on a user, such as by an imaging sensor or IR camera, may be processed by a processor and then presented to the user before one or more liquids may be emitted from a bidet head 218. The user is required in some embodiments, to approve the receiving of one or more liquids onto an exposed portion of their skin after detection of a hot spot or other health issue. In some embodiments, data collected and processed may lead to an automatic emission of one or more liquids from the bidet head 218 onto the exposed surface of a user after detection of a hot spot or other health issue.

FIG. 2B illustrates a cross-sectional view of a portion of an analytical toilet 240 in operation, according to an embodiment of the disclosure. Analytical toilet 240 shows a user 242 sitting on the seat 204. Sensor 206 imbedded in seat 204 detects the presence of a user. In one embodiment, the bidet wand 214 may extend into the bowl 202 and under user 242 before or after an excretion event. An IR camera located on head 218 is then be engaged to emit IR radiation 244 to thermographically detect heat differentials, such as one or more hot spots 246 on the exposed surface of user 242. The IR camera may take a general thermal reading of the exposed surface of the user 242 and calculate an average surface temperature. If a location of the exposed surface of a user has a temperature of about 1-3° C. higher than the average surface temperature, then a liquid may be dispensed from the head 218 directly onto the hot spot. In a preferred embodiment, if a location of the exposed surface of a user has a temperature of 3° C. than the average surface temperature, then a liquid may be dispensed from the head 218 directly onto the hot spot.

FIG. 2C illustrates a cross-sectional view of a portion of an analytical toilet 280 in operation, according to an embodiment of the disclosure. FIG. 2C illustrates dispensing of a fluidic material from the head 218 after a hot spot is detected by the IR camera. This is illustrated in FIG. 2C by dotted line 248 which represents a stream of fluid emitted by head 218 towards a hot spot. Any fluid that does not adhere to the user may drain into the bowl 202. Once a fluid is dispensed the wand 214 may then retract into the rear of the bowl.

Head 218 on bidet wand 214 may be designed to emit a high pressure stream of water. In some instances, the high pressure stream of water may be used to clean the bowl, such as streaks of feces remaining after the bowl is flushed. The wand 214 may rotate while emitting the water to clean the bowl 202. In other instances, the high pressure stream of water may be used to comminute or macerate a sample of feces from a user. Comminution or maceration of the feces may be necessary for improved analysis, such as for the detection of one or more parasites.

FIG. 2D illustrates a cross-sectional view of a portion of an analytical toilet 290 in operation, according to an embodiment of the disclosure. A user 242 excretes a sample of feces 292 in the bowl 202. The bidet wand 214 extends from the rear of the bowl 202 over the fecal shelf 208 where the feces 292 is located. A high pressure stream of water 294 may then be emitted from the bidet head 218 comprising a water outlet to comminute or macerate the feces 292. In other embodiments, a stream of fluid comprising one or more of a dye, tracer, or markers may be emitted from the bidet head 218 to prepare the feces sample for further analysis and detection of one or more biomarkers.

In some embodiments, the bidet wand 214 and head 218 may be adapted to detect one or biomarkers that are enhanced by ingestion of a dye, tracer, or marker by a user before excretion of a sample of feces. Detection of the one or more biomarkers may be carried out by an imaging sensor, such as for parasites.

Head 218 on bidet wand 214 may be designed to emit one or more flashes of ultra-violet (UV) light. The UV light is used to clean, disinfect, and sanitize the bowl. For example, UV light may be used to destroy E. coli bacteria or coronaviruses, such as SARS-CoV-1 or SARS-CoV-2 viruses.

Head 218 on bidet wand 214 may be designed to self-clean. In some instances, the bidet wand 214 or head 218 may have a residue from a liquid dispensing event or splash from the toilet. The head 218 may be designed to slowly emit or ooze water, soap, or other surfactant fluid to rinse any unwanted residues away. The head may also be capable of self-heating to drive off any residue.

### EXAMPLE

### Example 1. Detection and Treatment of a Skin Rash in an Analytical Toilet

Example 1 is illustrative of a preferred method of detecting a rash on the exposed surface of a user and treating the rash with an ointment. The method comprises:
1) A user sits on the seat of an analytical toilet.
2) A sensor in the seat detects the presence of the user.
3) The bidet head comprising an LED illumination source, camera and an infrared (IR) sensor extends into the bowl and under the user.
4) The LED illumination source emits light to illuminate the exposed surface of the user, and the camera in the bidet wand takes a three-dimensional image of the exposed surface of the user.
5) The infrared sensor detects the average skin temperature of the exposed surface imaged by the camera.
6) The infrared sensor detects localized elevated temperatures of ≥3° C. higher than the average temperature of the exposed surface determined in the previous step.
7) A 1 mL sample of a liquid is emitted from the bidet head comprising a mixture of anti-microbials: 5 mg of Neomycin Sulfate, 400 units of Bacitracin Zinc, and 5000 units of Polymyxin-B Sulfate.
8) The bidet head sprays 1 mL of a liquid comprising 5% by weight of lidocaine ointment.
9) The bidet head emits a stream of air at 15 psi for a period of 15 seconds.
10) The bidet wand and bidet head retracts into the back of the bowl.
11) The bowl is flushed to remove any debris remaining in the toilet.

The invention has been described with reference to various specific and preferred embodiments and techniques. Nevertheless, it is understood that many variations and modifications may be made while remaining within the spirit and scope of the invention.

What is claimed is:

1. A toilet comprising:
a seat for supporting a user during use of the toilet;
a bowl for catching excreta from the user; and
a retractable bidet wand adapted to extend after excretion has concluded;
a bidet head located on the end of the bidet wand, adapted to spray water on the exposed surface of the user;
an image sensor on the bidet wand, adapted to capture images of the exposed surfaces of the user; and
a processor to analyze the images to detect health issues.

2. The toilet of claim 1 wherein the bidet head is adapted to aim pressurized jets of water to remove debris from the bowl.

3. The toilet of claim 1 wherein the bidet head is adapted to aim pressurized jets of water at the excreta to comminute the excreta.

4. The toilet of claim 1 wherein the bidet head is adapted to capture gases in the bowl for analysis following excretion by a user.

5. The toilet of claim 1 wherein the bidet head is adapted to capture images of the excreta.

6. The toilet of claim 1 wherein the image sensor is a charge coupled device (CCD) or a complementary metal-oxide semiconductor (CMOS) sensor.

7. The toilet of claim 1 wherein the bidet wand and bidet head are capable of rotating around a longitudinal axis.

8. The toilet of claim 1 wherein the bidet head comprises an infrared (IR) camera.

9. The toilet of claim 8 wherein the infrared camera is adapted to identify heat differentials in the exposed surfaces of the user.

10. The toilet of claim 1 wherein the bidet head is adapted to emit a health and wellness spray towards the exposed surface of a user.

11. The toilet of claim 10 wherein the health and wellness spray comprises one or more ointments, lubricants, herbals, medicinal drugs, pain killers, topicals, essential oils, soaps, foams, cleansers, surfactants, moisturizers, deodorants, dyes, conditioners, or a combination thereof.

12. The toilet of claim 1 wherein the bidet head is adapted to emit a stream of air to dry the exposed surface of a user.

13. The toilet of claim 1 wherein the bidet head is adjustable to direct water or a health and wellness spray to the genitalia area of a male or female user.

14. The toilet of claim 1 wherein the bidet head is adapted to detect a fecal biomarker that is enhanced by ingestion of a dye, tracer, or marker by a user before excreting a sample of feces.

15. The toilet of claim 1 wherein the bidet head is adapted to spray a dye, tracer, or marker onto the surface of a sample of excreta to detect one or more biomarkers.

16. The toilet of claim 1 wherein the bidet head further comprises one or more image sensors capable of forming a three-dimensional image of the exposed surfaces of the user.

17. The toilet of claim 1 wherein the bidet head further comprises a light emitter to illuminate the exposed surface of a user.

18. The toilet of claim 1 wherein the bidet head comprises an ultra-violet (UV) light emitter to disinfect the bowl.

19. The toilet of claim 1 wherein the bidet head further comprises a self-cleaning device.

20. The toilet of claim 1 wherein the bidet head is adapted to heat an emitted liquid.
